(19) United States
(12) Patent Application Publication
(10) Pub. No.: US 2009/0287161 A1
(43) Pub. Date: Nov. 19, 2009

(54) METERED, MULTIPLE DOSE/ALIQUOT SYRINGE

(75) Inventors: Scott L. Traub, Wilbraham, MA (US); Gregory F. Brooks, Irvine, CA (US)

Correspondence Address: ALLERGAN, INC., 2525 DUPONT DRIVE, T2-7H, IRVINE, CA, 92612-1599, US

(73) Assignee: ALLERGAN, INC, Irvine, CA (US)

(21) Appl. No.: 12/121,000

(22) Filed: May 15, 2008

(51) Int. Cl. A61M 5/168

(52) U.S. Cl. 604/208

(57) ABSTRACT

A metered multiple aliquot/dose syringe includes a barrel having an open end and a opposing spaced apart port adapted to receive a needle. A piston is provided and slidably disposed within the barrel through the barrel open end and a push rod is connected to the piston for sliding the pistol within the barrel. Structure interconnected to the barrel and the push rod is provided for enabling stepwise movement of the push rod and the piston within the barrel and concomitant tactile and sound indicator of push rod and piston movement. A syringe within the scope of our present invention permits injection of accurate doses due to the physical stops or dosage administered indicators (i.e. elements 58 in FIG. 1, 22 in FIG. 6, 138 in FIG. 8, and elements 68 in FIG. 10) which can act to prevent hydraulic momentum from continuing to deliver fluid after thumb pressure is lifted off the push rod (piston).

There are many types of syringes available for medical (injections, instillations, etc.) and non-medical (basting/cooking, adhesive, lubrications, other industrial/manufacturing) uses. Over the years, there have been many enhancements to the basic syringe. These include, but are not limited to syringes that are disposable, include luer locks, have safety mechanisms to minimize needle sticks, are dedicated to medication cartridge systems (such as Carpojects®), as well as syringes that are factory pre-filled with a unit dose/aliquot of medication or other fluid.

With most syringes, by applying pressure to the piston, and controlling where the piston stops relative to tick marks indicating volumes delivered, the operator can administer multiple aliquots (equal or unequal) of fluid from the same syringe in order to deliver a predetermined total amount of fluid to a target location.

However, this takes good hand-to-eye coordination. In certain applications, such as delivery of multiple doses of medication sequentially during the same patient visit, it can be important to deliver very accurate amounts of fluid in a quick, convenient, precise and accurate manner. The proposed syringe accomplishes this goal and allow the operator to concentrate on other important aspects of the associated procedures.

### SUMMARY

A metered, multiple aliquot/dose syringe in accordance with the present invention generally includes a barrel having an open end and a opposing spaced apart port adapted to receive a needle. A piston is provided and slidably disposed within the barrel through the barrel open end and a push rod is connected to the piston for sliding the piston within the barrel.

In accordance with the present invention, structure is provided which interconnects the barrel and the push rod for enabling stepwise movement of the push rod and the piston within the barrel. This structure further provides concomitant tactile and/or an audible (sound) indication of such stepwise movement, thereby enabling the user to operate the syringe in delivering multiple doses without the visual observance thereof.

Thus, a syringe within the scope of our present invention permits injection of accurate doses due to the physical stops or dosage administered indicators (i.e. elements 58 in FIG. 1, 22 in FIG. 6, 138 in FIG. 8, and elements 68 in FIG. 10) which can act to prevent hydraulic momentum from continuing to deliver fluid after thumb pressure is lifted off the push rod (piston).

More particularly, the structure in accordance with one embodiment of the present invention may include the plurality of spaced apart ridges on an outer surface of the push rod and an engageable ridge disposed on an inner surface of the barrel.

In another embodiment of the present invention, the structure comprises a plurality of spaced apart detents in an outer surface of the push rod and a corresponding engageable ridge disposed on an inner surface of the barrel.

Still another embodiment of the present invention, the structure comprises a plurality of spaced apart ridges disposed on an outer surface of the barrel and an arm connected to the push rod. The arm includes a ridge disposed for stepwise engagement of the spaced apart ridges. More particularly, the spaced apart ridges may be aligned with one another only a portion of the barrel circumference and the arm may have a width smaller than a push rod circumference.

Yet another embodiment of the present invention, the structure includes a plurality of spaced apart ridges disposed on an outer surface of the barrel and a sleeve is provided which surrounds the push rod and includes a ridge disposed on an inner surface of the sleeve for stepwise engagement of the spaced apart ridges. More specifically, in this embodiment, the spaced apart ridges are circumferential about the barrel and the sleeve ridge is circumferential about the sleeve inner surface.

A further embodiment in accordance with the present invention includes structure which comprises a plurality of spaced apart detents disposed on an outer surface of the barrel and an arm connected to the push rod with the arm including a ridge disposed for a stepwise engagement of the spaced apart detents. More particularly, in this embodiment, the spaced apart detents may be aligned with one another over a portion of the barrel structure and the arm has a width smaller than a push rod circumference.

An additional embodiment of the present invention provides for a syringe in which the structure comprises a plurality of spaced apart detents disposed on an outer surface of the barrel and a sleeve surrounding the push rod includes a ridge disposed on an inner surface of the sleeve for stepwise engagement of the spaced apart detents. More particularly, in this embodiment, the spaced apart detents may be circumferential about the barrel and the sleeve ridge may be circumferential about the sleeve inner surface.

### DRAWINGS

The advantages and features of the present invention will be better understood by the following description when considered in conjunction with the accompanying drawings, in which:

FIG. 1 illustrates a metered, multiple aliquot-dose syringe in accordance with the present invention which generally includes a barrel, a piston slidably disposed therein along with a push rod connected to the piston and a structure interconnected to the barrel and the piston for enabling stepwise movement of the push rod and the piston within the barrel, more specifically, the structure may include a plurality of spaced apart ridges on an outer surface of the piston and an engageable ridge disposed on an inner surface of the barrel;

FIG. 2 is an illustration of the use of the present invention with a cystoscope for visualizing and performing procedures in a bladder while at the same time utilizing tactile and sound features of the present invention;

FIGS. 3, 4, and 5 illustrate alternative ridge and detent configurations encompassed by the present invention;

FIG. 6 is a plan view of a alternative embodiment of the present invention showing a barrel having a plurality of spaced apart ridges aligned with one another over a portion of the barrel;

FIG. 7 is a cross sectional view of the barrel illustrated in FIG. 6 further showing an arm connected to the push rod with the arm including a ridge disposed for a stepwise engagement of the spaced apart ridges on the barrel;

FIG. 8 is a plan view of an alternative embodiment of the present invention in which the barrel includes circumferential ridges and a sleeve connected to the push rod includes a ridge disposed on an inner surface of the sleeve for a stepwise engagement of the spaced apart ridges;

FIG. 9 is a cross sectional view of the embodiment shown in FIG. 8;

FIG. 10 is a plan view of yet another embodiment of the present invention illustrating a barrel having a plurality of spaced apart detents in an outer surface thereof along with a sleeve having a circumferential ridge for engaging the detents;

FIG. 11 is a cross sectional view of the embodiment shown in FIG. 9;

FIG. 12 is a plan view of still another embodiment of the present invention utilizing a barrel having a plurality of spaced apart circumferential detent in a barrel and a corresponding ridge formed in a sleeve surrounding a push rod; and

FIG. 13 is a cross sectional view of the embodiment shown in FIG. 12.

### DESCRIPTION

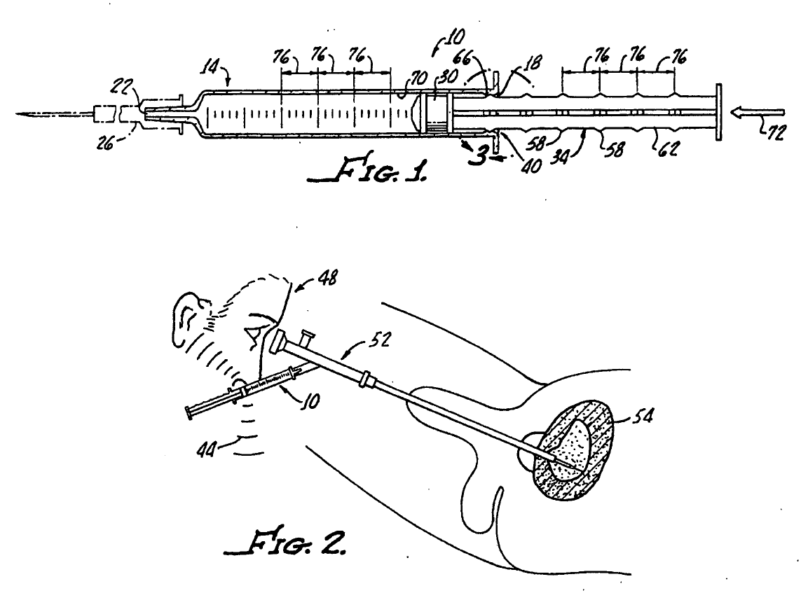

With reference to FIG. 1, there is shown a syringe 10 in accordance with the present invention for providing a metered multiple aliquot/dose that includes a barrel 14 having an open end 18 and an opposing spaced apart port 22 which is adapted for receiving a conventional needle 26, shown in broken line. A piston 30 is slidably disposed within the barrel 14 through the barrel open end 18 and a push rod 34 is connected to the piston for sliding the piston 30 within the barrel 14.

Structure 40 interconnecting the barrel 14 and the push rod 34 enables stepwise movement of the push rod 34 and the piston 30 within the barrel 14.

As will be described hereinafter in greater detail, the structure 40 is configured for producing sound indication as indicated by lines 44, in FIG. 2 of stepwise movement of the push rod 34 and piston 30 within the barrel 14 to enable an operator 48 to deliver multiple doses of a medicament without visual observation of the syringe 10. This is particularly useful in the delivery of a drug using an endoscope 52 as shown in FIG. 2.

As the operator 48 such as a surgeon or urologist) is busy manipulating the scope 52 so that he/she can visualize the proper area of the body tissue 54, he/she does not have to either have an assistant to control the syringe 10 or first place and secure the scope 52, and then let go of the scope 52 with one hand to grasp then push the syringe 10.

During this time, the scope 52 and needle 10 have a tendency to move. In prior art discoveries, the hydraulic momentum of the initial piston (not shown) push, may cause the piston to move past a desired point on a barrel (not shown), leading to too much fluid being administered at each injection site. The syringe 10 in accordance with the present invention prevents this over-run by having “ratcheted stops” spaced at proper distance for the desired volume to be delivered. It will further enable the procedure to be conducted by one person without the need for an assistant as the endoscopic operator 48 will not have to take his eyes off the image being relayed through the scope 52.

With reference again to FIG. 1, in accordance with the present invention, the structure 40 includes a plurality of spaced apart ridges 58 disposed on an outer surface 62 of the push rod 34 and an engagable ridge 66 disposed on an inner surface 70 of the barrel 14. Movement of the push rod 34 in a direction indicated by arrow 72 causes stepwise engagement between the ridges 58, 66 resulting not only in a tactile sensation by the operator 48 but also in sound generation as indicated at lines 44 in FIG. 2. Selected spacing 76 may provide for one milliliter doses, as an example, to be ejected from a multi-milliliter syringe. Thus, the syringe 10 in accordance with the present invention provides for an accurate and precise dose/aliquot of fluid administered in a fast and stepwise convenient manner. An enlargement of the structure 40 showing a ridges 58, 66 during engagement is illustrated in FIG. 3.

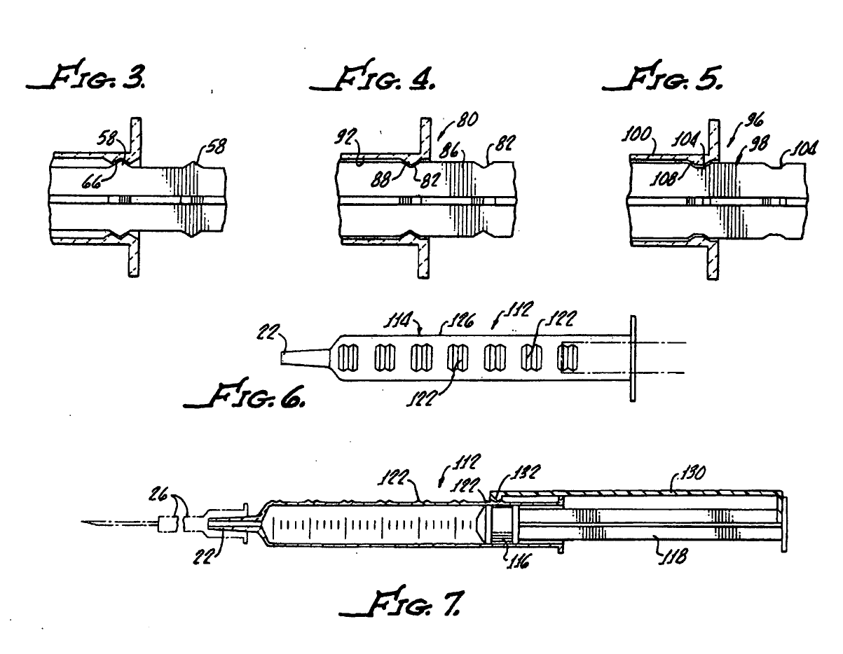

FIG. 4 is an alternative embodiment of the structure 80 in accordance with the present invention, wherein structure 80 comprises a plurality of detents 82 in a push rod outer surface 86 and a correspondingly engageable ridge 88 disposed on a barrel inner surface 92.

Control of the tactile sensory indication of stepwise movement provided by a structure 96 for a push rod 98 and barrel 100 is shown in FIG. 5 wherein a detents 104 and ridge 108 have a smoother contour than a corresponding ridges 82, 88 shown in FIG. 4 thereby changing a tactile and auditory sensing of movement of the push rod 98 within the barrel 100.

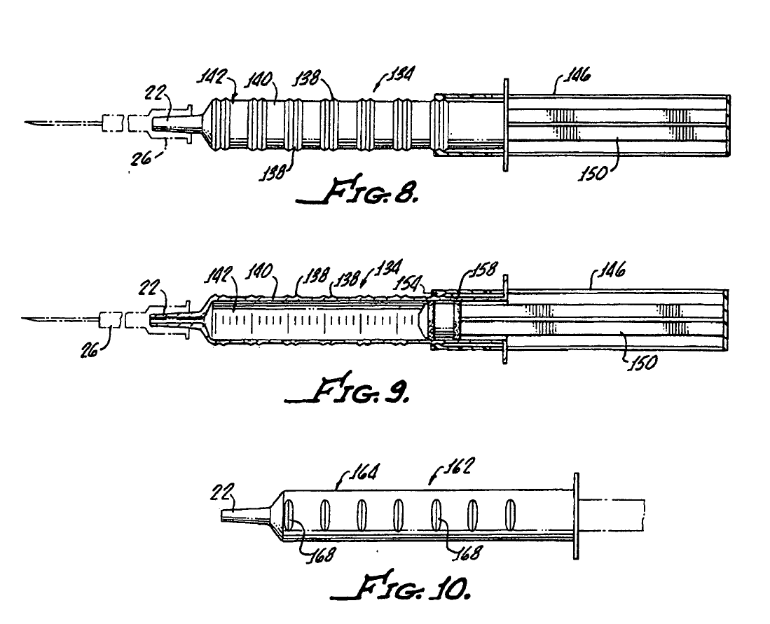

In yet another embodiment syringe 112 is illustrated in FIGS. 6 and 7 which includes a barrel 114, piston 116, and a push rod 118. The syringe barrel 114, as illustrated in FIGS. 6 and 7, includes a plurality of spaced apart ridges 122 disposed on an outer surface 126 of the barrel 114 and an arm 130 connected to a push rod 118 which includes an arm ridge 132 for a stepwise engagement with the ridges 122. In this syringe 112, the ridges 122 are aligned with one another over a portion of the barrel 114 circumference, or outer surface, 126 and the arm 130 has a width smaller than a circumference of the push rod 118.

In yet another embodiment syringe 134 is illustrated in FIGS. 8 and 9 with common character references indicating identical or substantially similar elements as hereinbefore discussed in connection with other embodiments of the present invention.

As shown in FIGS. 8 and 9, the syringe 134 includes a plurality of spaced apart circumferential ridges 138 disposed on an outer surface 140 of a barrel 142, and a sleeve surrounds and is connected to a push rod 150. A circumferential ridge 154 disposed on an inner surface of the sleeve 146 enables stepwise engagement of the barrel ridges 138 in a manner as hereinabove described in connection with earlier described embodiments of the present invention.

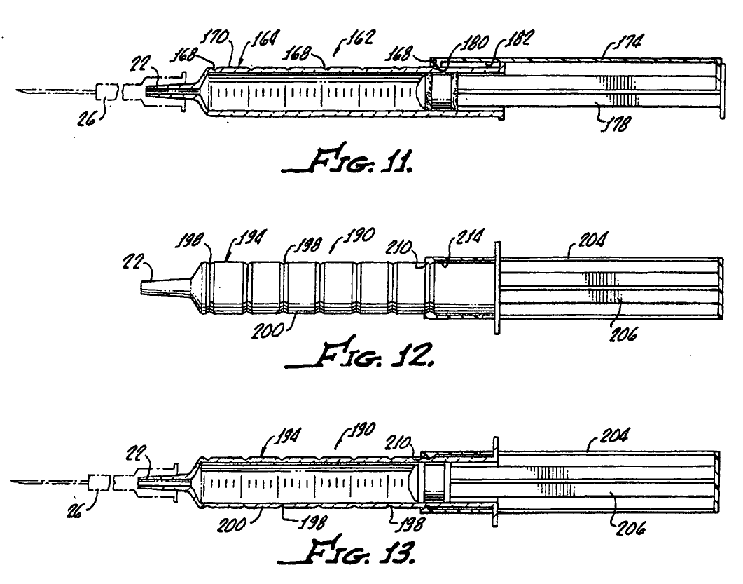

FIGS. 10 and 11 illustrate yet another embodiment syringe 162 in accordance with the present invention that includes a barrel 164 which includes a plurality of spaced apart detents 168 and an outer surface 170 of the barrel 164 and an arm 174 attached to a push rod 178 and includes a ridge 180 disposed on an arm inner surface 182 for a stepwise engagement with the detents 168 in a stepwise manner.

Still another embodiment syringe 190 is shown in FIGS. 12 and 13 which includes a barrel 194 having spaced apart circumferential detents 198 in a barrel outside surface 200 and a sleeve 204 surrounding a push rod 206 includes a ridge 210 disposed on an inside surface 214 of the sleeve 204 for stepwise engagement of the spaced apart detents 198.

A method in accordance with the present invention utilizes any one of the syringes 10, 112, 134, 162, 190 hereinabove described and includes with respect to syringe 10 disposing a medicament in the barrel 14 and operating the structure 40 interconnecting the barrel 114 and the push rod 34 to provide stepwise movement of the push rod 34 and piston 30 within the barrel 114 in order to administer metered multiple aliquot/doses of medicament.

### EXAMPLE

The present invention is especially useful with a cystoscope for injecting a pharmaceutical, such as botulinum toxin, into a target tissue, such as a bladder wall to treat a disease or condition such as a urological disease or condition (i.e. a bladder dysfunction such as overactive bladder), a prostate disorder, an ocular disease or condition or any other human ailment, condition or disease.

The normal micturition process is a result of a complex network of innervation of the bladder and urethral sphincter that ensures satisfactory bladder filling followed by timely voiding in healthy individuals. In order to achieve storage of urine in the bladder during the filling phase, the bladder neck and urethra remain closed and the detrusor muscle is relaxed (via stimulation of the noradrenalin beta receptors in the dome of the bladder). In the healthy bladder, when the pressure within the bladder is greater than that within the urethra, urination begins. The sensations of pain and bladder fullness are carried by the afferent fibers, which relay their message from the bladder to the micturition center in the pons of the brain triggering micturition. The voiding phase involves coordinated relaxation of the base of the bladder and urinary sphincter (via stimulation of the noradrenalin alpha receptors) and contraction of the detrusor muscle in the bladder wall secondary to inhibition of noradrenergic stimulation followed by parasympathetic stimulation via the neurotransmitter acetylcholine.

Overactive bladder is a condition resulting in a disruption to the normal micturition process. It is a syndrome complex characterized by urinary urgency, frequency and may or may not be accompanied by incontinence. Incontinence is due to involuntary contraction of the detrusor muscle during bladder filling (detrusor overactivity). Most cases of incontinence arise without obvious pathology. In such cases, abnormal detrusor contractions are termed idiopathic bladder overactivity. A smaller number of cases are secondary to neurogenic pathology and are termed neurogenic detrusor overactivity.

### Neurogenic Detrusor Overactivity

The pathophysiology of OAB is complex, involving peripheral and central nervous system (CNS) dynamics. Several CNS disorders are associated with the development of OAB, including spinal cord injury and multiple sclerosis. Neurological disease involving the spinal cord can result in incontinence secondary to a loss of inhibitory input from the micturition center and from interruption of the spinobulbospinal pathways which normally control bladder behavior. In the event of a spinal cord lesion, a change of balance of the effects of the afferent fibers, located between the muscle and submucosa of the bladder, is seen. The unmyelinated C fibers become functionally dominant and the detrusor hyperreflexia described in such patients is considered due to the reflex mediated by these unmyelinated C fibers.

The result, demonstrable on urodynamic evaluation, is abnormal involuntary detrusor contractions, often leading to incontinence. In addition, such patients frequently suffer from loss of coordinated relaxation of the urethral sphincters that normally precedes micturition. This lack of coordinated activity can result not only in incontinence but also in vesico-ureteric reflux which, if left untreated, can lead to potential renal damage.

### Available Treatments

Clean intermittent self-catheterization (CIC) is commonly used to drain the bladder, manage neurogenic incontinence and prevent vesico-ureteric reflex. When employing CIC, the patient inserts a catheter via the urethra into the bladder in order to void urine. CIC, however, can be associated with infection, which can exacerbate the problem of urinary incontinence and, in some circumstances, lead to renal damage. Common pharmacologic treatments to reduce bladder contractility include anticholinergics, antispasmodics and tricyclic antidepressants. However, these therapies are associated with a high incidence of side effects. Side effects of anticholinergics include dry mouth, constipation and blurred vision. Currently, the only options available to patients who do not respond to or discontinue anticholinergic therapy are invasive procedures such as implantable devices to chronically stimulate the sacral nerve or surgical bladder augmentation. While these procedures may be effective for some patients, they are highly invasive, do not necessarily guarantee continence, and may have long term complications.

### BOTOX® (Botulinum Toxin Type A Purified Neurotoxin Complex) Treatment

Recently, studies have been carried out using BOTOX® (botulinum toxin) in the treatment of patients who suffer from bladder overactivity. Suppression of involuntary detrusor contractions has been attempted via the local administration of BOTOX® (botulinum toxin) to the detrusor muscle, which inhibits acetylcholine release by cleaving SNAP 25, a protein integral to successful docking and release of vesicles within the nerve endings, including acetylcholine, calcitonin gene-related peptides (CGRP), glutamate and substance-P. BOTOX® (botulinum toxin) is believed to inhibit the acetylcholine mediated detrusor contractions and may also inhibit other vesicle-bound neurotransmitters in both the afferent and efferent pathways of the bladder wall, urothelium or lamina propria.

There is evidence for the successful use of BOTOX® (botulinum toxin) in the management of neurogenic incontinence. It has been shown that botulinum toxin injections of 200 U to 300 U into the detrusor across 20 to 30 injection sites (10 units per mL per injection site) have been effective in restoring continence and enabling reduction or cessation of anticholinergic medication in such patients. In one study enrolling 21 patients, 17 of the 19 patients with follow-up data had restored continence within 6 weeks. To date, treatment of over 900 neurogenic overactive bladder patients with BOTOX® (botulinum toxin) at doses ranging from 200 U to 300 U in 20 to 30 injection sites has been reported. Treatment benefit has been described to last between 6 and 12 months with an acceptable side effect profile.

Endoscopy of the urinary bladder via the urethra is cystoscopy. Diagnostic cystoscopy is usually carried out with local anesthesia. General anesthesia is sometimes used for operative cystoscopic procedures.

When a patient has a urinary disease or condition, a physician can use a cystoscope 52, see FIG. 2 to see inside of the bladder and urethra. The urethra is the tube that carries urine from the bladder to the outside of the body. The cystoscope has lenses which permit the physician to focus on the inner surfaces of the urinary tract. Some cystoscopes use optical fibres (flexible glass fibres) that carry an image from the tip of the instrument to a viewing piece at the other end. The cystoscope is as thick as a pencil and has a light at the tip. Many cystoscopes have extra tubes to guide other instruments for surgical procedures to treat urinary problems.

There are two main types of cystoscopy—flexible and rigid—differing in the flexibility of the cystoscope. Flexible cystoscopy is carried out using local anesthesia on both sexes. Typically, lidocaine gel (such as the brand name Instillagel) is used as an anesthetic, instilled in the urethra. Rigid cystoscopy can be performed under the same conditions, but is generally carried out under general anesthesia, particularly in male subjects, due to the pain caused by the probe. The embodiments of our invention set forth herein (see eg the Figures) can be used to accurately and precisely inject a metered dose (aliquots) of a botulinum toxin (such as BOTOX. DYSPORT, MYOBLOC, or XEOMIN) into the bladder wall (detrusor) of a patient to treat a bladder dysfunction. Our invention is not limited to use to treat a bladder dysfunction or to administration of a botulinum toxin, as it can be used for any therapeutic, cosmetic or research use in which accurate and precisely metered doses of an aqueous pharmaceutical is desired.

Although there has been hereinabove described a specific metered, multiple dose/aliquot syringe and method in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

1-4. (canceled)

5. A metered, multiple aliquot/dose syringe comprising:
a barrel having an open end and an opposing spaced apart port adapted to receive a needle;
a piston slidably disposed within said barrel through the barrel open end;
a push rod connected to said piston for sliding said piston within said barrel; and
structure interconnecting said barrel and said push rod for enabling stepwise movement of said push rod and said piston within said barrel and producing sound and/or tactile indication of said stepwise movement to enable operator of the syringe said structure comprising a plurality of spaced apart ridges disposed on an outer surface of said barrel and an arm connected to said push rod for movement exterior to said barrel, said arm including a ridge disposed for stepwise engagement of said spaced apart ridges disposed on the barrel outer surface.

6. The syringe according to claim 5 wherein said spaced apart ridges are aligned with one another over a portion of a barrel circumference.

7. The syringe according to claim 6 wherein said arm has a width smaller than a push rod circumference.

8. A metered, multiple aliquot/dose syringe comprising:
a barrel having an open end and an opposing spaced apart port adapted to receive a needle;
a piston slidably disposed within said barrel through the barrel open end;
a push rod connected to said piston for sliding said piston within said barrel, and
structure interconnecting said barrel and said push rod for enabling stepwise movement of said push rod and said piston within said barrel and producing sound and/or tactile indication of said stepwise movement to enable operator of the syringe in delivery of multiple doses of medicament without visual observance of the syringe, said structure comprising a plurality of spaced apart ridges disposed on an outer surface of said barrel and a sleeve surrounding said barrel and push rod and including a ridge disposed on an inner surface of said sleeve for stepwise engagement of said spaced apart ridges disposed on the barrel outer surface.

9. The syringe according to claim 8 wherein said spaced apart ridge are circumferential about said barrel.

10. The syringe according to claim 9 wherein the sleeve ridge is circumferential about the sleeve inner surface.

11. A metered, multiple aliquot/dose syringe comprising:
a barrel having an open end and an opposing spaced apart port adapted to receive a needle;
a piston slidably disposed within said barrel through the barrel open end;
a push rod connected to said piston for sliding said piston within said barrel, and
structure interconnecting said barrel and said push rod for enabling stepwise movement of said push rod and said piston within said barrel and producing sound and/or tactile indication of said stepwise movement to enable operator of the syringe in delivery of multiple doses of medicament without visual observance of the syringe, said structure comprising a plurality of space apart detents disposed on an outer surface of said barrel and an arm connected to said barrel and push rod, said arm including a ridge disposed for stepwise engagement of said spaced apart detents disposed in the barrel outer surface.

12. The syringe according to claim 11 wherein said spaced apart detents are aligned with on another over a portion of a barrel circumference.

13. The syringe according to claim 12 wherein said arm has a width smaller than a push rod circumference.

14. A metered, multiple aliquot/dose syringe comprising:
a barrel having an open end and an opposing spaced apart port adapted to receive a needle;
a piston slidably disposed within said barrel through the barrel open end;
a push rod connected to said piston for sliding said piston within said barrel, and
structure interconnecting said barrel and said push rod for enabling stepwise movement of said push rod and said piston within said barrel and producing sound and/or tactile indication of said stepwise movement to enable operator of the syringe in delivery of multiple doses of medicament without visual observance of the syringe, said structure comprising a plurality of spaced apart detents disposed on an outer surface of said barrel and a sleeve surrounding said barrel and push rod and including a ridge disposed on an inner surface of said sleeve for stepwise engagement of said spaced apart detents disposed on the barrel outer surface.

15. The syringe according to claim 14 wherein said spaced apart detent are circumferential about said barrel.

16. The syringe according to claim 15 wherein the sleeve ridge is circumferential about the sleeve inner surface.

17. (canceled)
